(19) United States
(12) Patent Application Publication
(10) Pub. No.: US 2010/0160990 A1
(43) Pub. Date: Jun. 24, 2010

(54) FIRST AID SYSTEM, PROCEDURE FOR ITS OPERATION, AND PORTABLE FIRST-AID DEVICE FOR USE ESPECIALLY IN THE SYSTEM

(76) Inventor: András Gótzy, Budapest (HU)

Correspondence Address: Olson & Cepuritis, LTD., 20 NORTH WACKER DRIVE, 36TH FLOOR, CHICAGO, IL, 60606, US

(21) Appl. No.: 12/449,808

(22) PCT Filed: Feb. 25, 2008
(86) PCT No.: PCT/HU2008/000021

§ 371 Date: Jan. 11, 2010

(30) Foreign Application Priority Data
Feb. 28, 2007 (HU) P 07 00182
Feb. 20, 2008 (HU) P 08 00111

(51) Int. Cl. A61N 1/39; B65D 71/00

(52) U.S. Cl. 607/5; 206/570

(57) ABSTRACT

The invention relates to a first aid application system, which has at least one installed unit (1) containing first-aid tools and a device (devices) for forwarding information, which installed unit (1) is in communicating connection with a centre (2) receiving and forwarding information concerning first aid application. The system is characterised by that the information mediating device(s) of the installed unit (1) is (are) constructed to be suitable for displaying advertisements, announcements or other, for example public information other than first aid information, or/and they also contain a tool for this purpose. Furthermore the installed unit (1) has at least a defibrillator (7) and a breathing apparatus (6) as first-aid tools. The procedure according to the invention is characterised by that in the case of an accident or/and indisposition information is forwarded from the installed unit (1) to the centre (2) about the event; and on the basis of the information received from the centre (2) the person(s) suffering an accident or taken by an indisposition is (are) attended to—applied first aid—on the site, if necessary. During the period between the application of first aid and the next similar application advertisements or other information is provided continuously or with interruptions using the installed unit. The portable first-aid tool is characterised by that it contains at least a breathing apparatus (6) and a defibrillator (7).

The invention relates to a first aid system, which has at least one installed unit containing first-aid tools and tool(s) for forwarding information, which unit communicates with a centre receiving and forwarding first aid information. The invention also relates to a procedure for the operation of this system, and to a portable first aid device that can be used especially in said system.

People can be taken by an indisposition or accidents can happen to them practically anywhere, under any circumstances. Often the chances of survival or recovery without any permanent health damage depend on how quickly patients receive appropriate treatment. Generally the first, often life-saving phases of such treatment are performed by persons currently staying near the patient, who are unskilled from a medical aspect and need to rely on their own knowledge—which is obviously unsatisfactory in most cases—until the doctor arrives.

Interventions made without special knowledge/experience (or the lack of interventions) may result in severe, or even fatal effects even beside the best intentions. Therefore people working/staying at places where accidents are more likely to happen (e.g.: people working in plants, vehicle drivers, etc.) are obliged to take part in first aid courses of different levels, and first aid kits containing a few basic tools are kept at such places.

Recently there has been a tendency of installing defibrillators at busy places, which devices have proved to be very useful several times in the hands of skilled people. However, it must be pointed out that the improper use of a defibrillator by a person unskilled from this aspect may result in serious damage or even death.

At the same time in most accidents or in most cases of indisposition having participated in a training course or having first aid kits or other tools at hand does not ensure a satisfactory level of intervention, as specialist skills are still missing and they are not put in use until the doctor (rescue team) arrives.

Interventions performed by unskilled persons or persons with little special knowledge (or the lack of intervention) involve several sources of danger. Namely:

a) such persons may be afraid of making an intervention as they are inexperienced, and they are afraid of doing “something wrong”, so they do not do anything to provide help or they perform an act with the intention to help only at an insufficient level;

b) they do not recognise the problem or problems or they make a mistake and choose the wrong tools needed for the intervention or determine the order of the acts of intervention wrongly;

c) they do not use the available first-aid tools or they use them wrongly.

A solution aimed at overcoming the above disadvantages at least partly is described in patent specification no. DE 103 52 210 A1, the subject of which relates to emergency equipment used for requesting help and for rendering first aid basically in the case of road accidents. This equipment contains a rescue unit and an information and communication unit, and via the latter unit telephone or television connection can be established with a certain base station from where the person rendering help at the accident—either the driver or the passenger of the vehicle—can receive instructions about attending to the person, e.g.: injured person who suffered the accident and information about when help, e.g.: the ambulance can be expected to arrive at the place of the accident.

The emergency equipment described above belongs to its owner—user—, first of all to a vehicle driver, and the appropriately small sized box containing the information/communication elements and first-aid tools—bandages are mentioned in the specification—is fixed in the driver's vehicle practically in a releasable way. As a result of this the applicability of the emergency equipment is restricted to the vehicle driver and/or the passenger/passengers on the one hand, and on the other hand it is not suitable for the treatment of heart or respiratory failures caused by the accident, because there is not enough space in the small sized box to accommodate medical devices of the size needed for such treatments.

Patent specification no. US 2005/0006109 A1 describes a system providing emergency treatment, which can be put into operation first of all in the case that fire, accidents, etc. occur in mostly multi-storied public buildings such as hospitals, educational facilities, transport stations (e.g.: airports, bus terminals, railway stations, etc.). The system is based on that in part-units of the facility, in each separate sub-unit—e.g.: on each level in a multi-storied building—tools needed for rescue work are placed, such as a fire extinguisher, alarm, defibrillator; communication device (telephone, computer communication system, etc.), and an internal central station communicating with the sub-units belongs to each unit, e.g.: to each multi-storied building, which central station is in communicating connection—e.g.: Internet, satellite, telephone connection—with a remote central rescue station, from where for example fire appliances, ambulance cars, etc. can be sent to the given location for emergency assistance in the case of fire or accidents. Consequently this is a system allocated to a certain public facility enabling rather complicated emergency assistance, basically aimed at the elimination of fire damage, although defibrillators exclusively are mentioned in the specification in a few lines as possible emergency devices.

Patent specification no. U.S. Pat. No. 6,472,988 B1 relates to a system for the remote inspection of fire extinguishing personal rescue devices and for controlling their operation, which is based on that a mobile unit is also connected to the compressed-air breathing apparatus worn by fire-fighters while extinguishing fires, which mobile unit is in permanent radio connection with a central remote control station, which intervenes into the operation of the breathing apparatus, if deviations endangering the fire-fighter are detected as compared to the basic data of the system.

The task to be solved with the invention is to provide a first-aid system for public use, with the help of which persons trained to operate the system who are not professional health workers; but even persons having no special skills at all or only minimal special skills can perform necessary first-aid interventions safely and efficiently in cases of indisposition, accidents or other events. Furthermore the task to be solved with the invention is on the one part to enable public use on public areas, to enable the establishment of first aid systems used as widely as possible in public facilities, e.g.: hospitals, other health, scientific, commercial, financial, etc. institutes, and on the other hand to ensure optimal conditions for efficient emergency life-saving interventions outside of hospitals, on the site of accidents or indispositions, first of all on public areas and also in public facilities.

The invention is based on the following recognitions:

the costs of the establishment, maintenance and operation of public first-aid sites and a centrally controlled high-standard system described for example in patent specification no. US 2005/006109 mentioned above would be so high that they could hardly be financed from state budget sources. However, if the system is established and operated in a way that during the periods in between the relatively rare cases of first aid application of a limited term the first-aid tools set up on public areas and public facilities are used for conveying advertisements, information, public and other announcements, the costs incurred in connection with the first aid system could be at least reduced, or even completely covered in a given case, and the system could be operated economically.

The invention is also based on the recognition that in the case of indisposition or other accidents resulting for example in the loss of consciousness, life-saving interventions are significantly more efficient, if a defibrillator and a breathing apparatus are used together, than in the case of using only one of these devices as described in patent specification no. US 2005/006109. It is known that during defibrillation a defibrillator is able to control the electric chaos occurring in the heart muscle. During defibrillation as a result of the electric current passing through the heart the cells of the heart muscle are depolarised at the same time, and if repolarisation also takes place at the same time, there is a chance of restoring normal heart rhythm. At the same time, among the factors influencing the efficiency of defibrillation, acidosis, hypoxia and electrolytes cannot be efficiently influenced only with a defibrillator, while the oxygen supply and the acid-base balance of the cells can be improved by artificial respiration, by adding oxygen, which is a dominant and critical factor, as the acidotic hypoxic heart muscle shows a slower response to defibrillation. However, if the cells are “prepared” by supplying oxygen to them, the efficiency of defibrillation is significantly more likely. Furthermore, artificial respiration performed at the same time as defibrillation also ensures appropriate tissular oxygenation.

In order to solve the set task, obviously communication tools for forwarding—or exchanging—information also need to be allocated to the first-aid tools set up on different locations far away from each other, where said communication tools are installed in one single unit with the first-aid tools, and with the help of said communication tools information can be forwarded to a remote skilled person—who is not staying on the given site and is not able to appear on the site in person urgently—practically to a doctor about the condition of the person who has suffered an accident or has been taken by an indisposition, and the skilled person can give professional instructions through said communication tool(s) to the person attending to the patient having no professional health skills in a given case. A separate information mediating device may be used for providing information from the site of the accident/indisposition on the one part, and a separate tool for providing information to the—generally unskilled—persons attending to the patient on the site of the accident/indisposition, needed for attending to the patient. In a system like this first aid can be applied to the patient at a higher professional level, increasing by this the chances of survival and recovery without permanent health damage.

Finally, another important recognition is that the system according to the invention can be realised in a way ensuring different service standards, depending on the currently available financial resources. If there is enough money, the installed unit may include a bag containing tools ensuring communication of maximum efficiency—a camera, screen, telephone; blood pressure monitor, ECG recorder, etc.—, a defibrillator, breathing apparatus and other first-aid tools and equipment. As a result of this resuscitation can be performed and first aid can be applied on a relatively large area in the region of the installed unit.

However, if because of financial limits or other aspects only the life-saving function is ensured only on a certain relatively small area used by a large number of people, e.g.: railway stations, shopping centres, sports fields, etc., it is enough to use an installed unit containing communication tools for keeping in contact with the centre—a screen and a camera—, but in its bag there is only a breathing apparatus and a defibrillator. In this case a person previously trained to use the tools must be allocated to the installed unit, and obviously this person can also receive information from the centre.

In both cases described above, when the installed unit is not life-saving/first aid application mode, on the screen of the installed unit advertisements, information, announcements can be displayed either continuously (within a given period) or with interruptions.

On the basis of the above recognitions the set task was solved with a first aid application system, which has at least one installed unit containing first-aid tools and a tool (tools) for forwarding information, which installed unit is in communicating connection with a centre receiving and forwarding information concerning first aid application; and which system is characterised by that
the information mediating device(s) of the installed unit is (are) constructed to be suitable for displaying advertisements, announcements or other, for example public information other than first aid information, or/and they also contain a tool for this purpose,
the installed unit has at least a defibrillator and a breathing apparatus as first-aid tools.

Favourably the centre is in communicating connection with several installed units.

The breathing apparatus can be manually operated, automatic or half-automatic; it is especially favourable to include an automatic breathing apparatus in the system according to the invention. In a given case the breathing apparatus also includes an oxygen tank or a tank filled with a mixture of gases containing oxygen, and an oxygen mask that can be placed on the patient's face. Such a breathing apparatus that can be included in the system according to the invention is known in trade by the name “Oxylator”; first of all the use of the type “Oxylator FR300” is favourable, which is automatic. The unit forming a part of the system according to the invention containing a defibrillator and a breathing apparatus forms a so-called “cardio-respiratory” system. The defibrillator may also be constructed to be suitable for providing direct information on the patient's condition, e.g.: ECG recordings to the centre or to the rescue unit, e.g.: to the ambulance car.

Another construction of the system is characterised by that as an information mediating device it has a display unit, practically a screen, favourably a flat screen, especially favourably a plasma screen or LCD screen; or a touch-screen and/or a camera, favourably a digital camera, especially favourably a CCD camera, a surveillance camera, and/or a wired or wireless telephone; and/or a microphone, especially a head microphone or outdoor monitoring microphone; and/or a loudspeaker; and/or a position finder, especially a global position finder, namely a GPS (Global Positioning System)—based position finder; and that as first-aid tools it contains bandages and/or a disinfectant; and/or EKG recorder; and/or blood pressure monitor; and/or a blood typing kit; and/or medicines, especially analgesics; and/or tools for cutting, especially scissors or/and a scalpel.

The installed unit of the system according to the invention can be regarded as a complex small first-aid station, which—beside filling other first-aid functions in a given case—provides a chance of survival with a maximum efficiency by restoring or restarting two basic life functions—respiration and blood circulation—simultaneously, interacting with each other, regarding and treating the heart and the lungs as one single unit. The information system favourably with a plasma screen—which shows the handling of the life-saving devices step by step—enables the optimal performance of the accident elimination/rescue and first aid activities. In the interest of this practically the centre is constructed to be suitable for receiving, forwarding, processing and—in a given case—storing information; and the installed unit has an information mediating device (devices) suitable for forwarding information automatically; and the information mediating device(s) of the installed unit are constructed to be suitable for providing information regarding the use of the first-aid tools. Due to automatic forwarding information about the accident/indisposition can be forwarded practically immediately to the centre or to the dispatcher/rescue unit, etc. Obviously the information about the use of the first-aid tools are basically for the unskilled persons staying on the site of the accident/indisposition ready to provide help.

In respect of the present invention, by first-aid tools, apart from the generally used meaning of the phrase, we mean all tools that may be used in stabilising the patient's condition, delaying the deterioration or facilitating the improvement of the patient's condition.

In respect of the present invention, by information mediating devices we mean all devices taking part in the flow of information between the installed unit and any other element of the system according to the invention and the person using the system in any way or being connected to the system, including producing, processing and forwarding information. In accordance with this information mediating devices are especially devices for producing and forwarding sound and picture information (for example cameras, microphones, screens, loudspeakers), positing finding devices, e.g. GPS (Global Positioning System) based device(s) and devices forwarding such data, diagnostics devices, such as blood pressure monitor, ECG recorder, and wired and wireless data transfer systems, etc. It is obvious for specialists that information mediating devices also include data carrier devices and data recording devices.

By site or the site of the accident we mean the environment of the installed unit within which there is a patient in need of treatment close enough to the installed unit so that the patient can receive first aid with the help of the installed unit, even by using its removable and portable tools. In the present description the phrase “site of the accident” is used to refer to the environment of the installed unit even if there is no actual accident or indisposition.

By centre we mean a part of the system according to the invention, which collects the information received from the individual installed units and gives further instructions to the installed units or other units forming a part of the system, e.g.: rescue units, in accordance with the information collected. The centre may be man-controlled or automatic.

A possible construction of the system is characterised by that the installed unit contains a tool (tools) suitable for mediating information, advertisements, emergency warnings and other announcements—practically from a centre—locally, within a given action radius in the environment of the installed unit, to mobile telephones, PDAs and similar devices, for example via SMS, MMS, etc. Furthermore it may be useful, if the installed unit contains a device providing WiFi access in its environment, within a given action radius.

The invention also relates to a procedure for operating the first aid system described above, which procedure is characterised by that
in the case of an accident or/and indisposition information is forwarded from the installed unit to the centre about the event;
on the basis of the information received from the centre the person(s) suffering an accident or taken by indisposition is (are) attended to on the site, if necessary;
during the period between the application of first aid and the next similar application advertisements or other information is provided continuously or with interruptions using the installed unit.

According to a favourable realisation of the procedure advertisements or other information are mediated using the same information forwarding device, especially a flat screen, favourably a plasma screen, LCD screen or touch-screen as used for displaying information concerning the application of first aid.

If for example in the environment of the installed unit there is an emergency situation deriving from an accident or indisposition involving the loss of consciousness, in order to start life functions or perform resuscitation—for the life-saving intervention—defibrillation and artificial respiration is applied at the same time. However, if a different type of accident/indisposition happens, for example if a wound needs to be bandaged, etc., the first-aid tools in the installed unit are used in the traditional way.

According to a further realisation of the procedure information is forwarded from the site of the accident/indisposition to the centre about the site of the accident and the patient's condition, and, in a given case, about the patient's age, sex, one or more identifiers; and a still or motion picture is mediated about the patient or/and about a part—e.g.: an injured part—of the patient's body; and, if necessary, information is provided about the patient's blood type, blood pressure and ECG recording.

By the patient's identifier we mean any data on the basis of which information can be obtained about the patient. Such identifier may be for example the personal identification number, the health insurance number, etc. The use of an identifier may be favourable, because in this way the data needed for attending to the patient may be available for the doctor or a similar specialist immediately, if they have access to the appropriate database. For example by using the health insurance identifier the doctor may obtain information immediately about the patient's blood type, sensitivity to drugs, etc.

The invention also relates to a portable first-aid device for use especially in the system, which device has tools for the application of first aid situated in the storing-carrying unit, and which is characterised by that it contains at least a breathing apparatus and a defibrillator. Practically this tool has an automatic breathing apparatus. It may also be favourable, if the defibrillator is constructed to be suitable for forwarding information concerning the patient's condition especially to a place where ECG tests are performed and the test results are evaluated.

The information forwarded to the doctor or a similar specialist can be forwarded only once or continuously. Obviously it is enough to send permanent data such as the patient's identifier only once. At the same time it is favourable, if continuously changing data describing the patient's condition is made available to the doctor continuously, such as data concerning blood pressure or ECG. In a given case the defibrillator itself may also be constructed to fill this function.

According to another especially favourable realisation of the procedure the information forwarded to the site of the accident is chosen from the following information, separately from each other: things to do to stabilise the patient's condition; method of using the available first-aid tools; questions to determine further tasks.

In accordance with a further criteria of the procedure, information is forwarded from the centre or through the centre to the site of the accident especially about stabilising the patient's condition and the method of using the available first-aid tools, especially the defibrillator and the breathing apparatus together or simultaneously. According to another method of realisation a rescue unit is sent from the centre or through the centre to the site of the accident/indisposition, which rescue unit is provided with the information received from the site of the accident needed for the treatment of the patient.

Below the accident is described in detail on the basis of the attached drawings showing the system according to the invention and construction examples of the system's installed unit. In the drawings

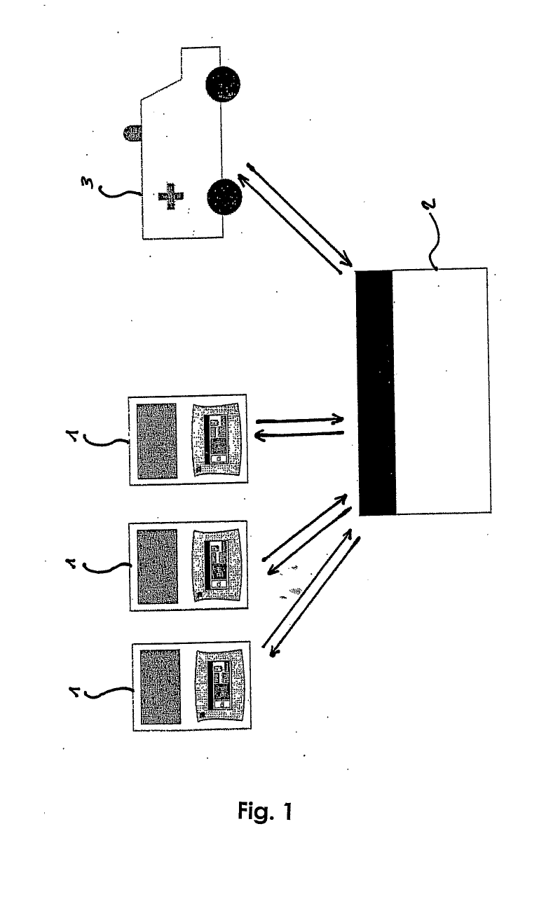

FIG. 1 is the schematic front view of an installed unit;

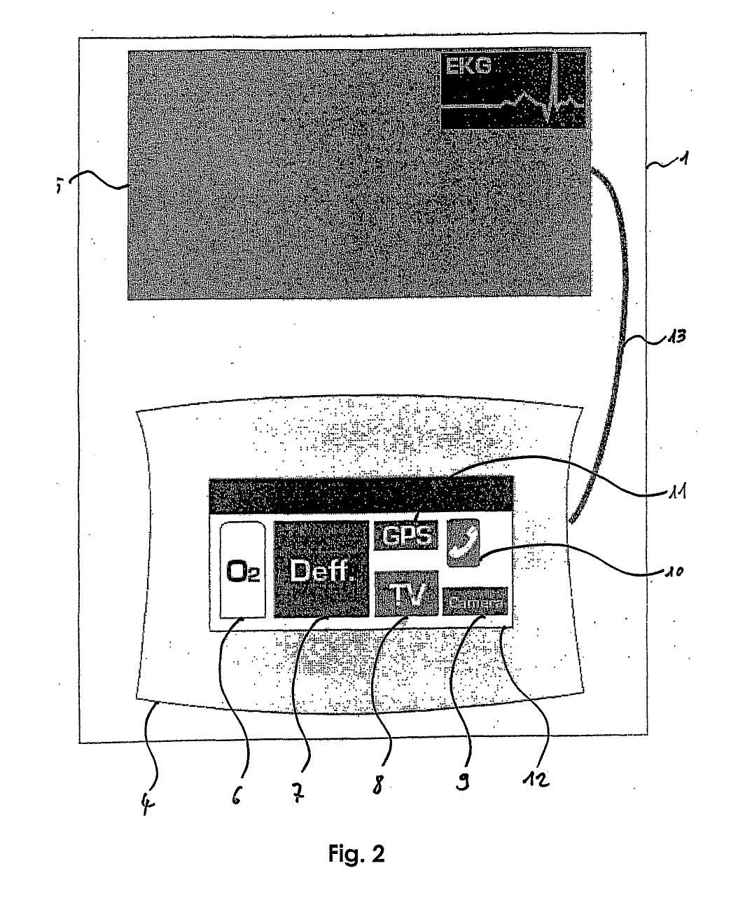

FIG. 2 is the schematic representation of the operation of the system according to the invention;

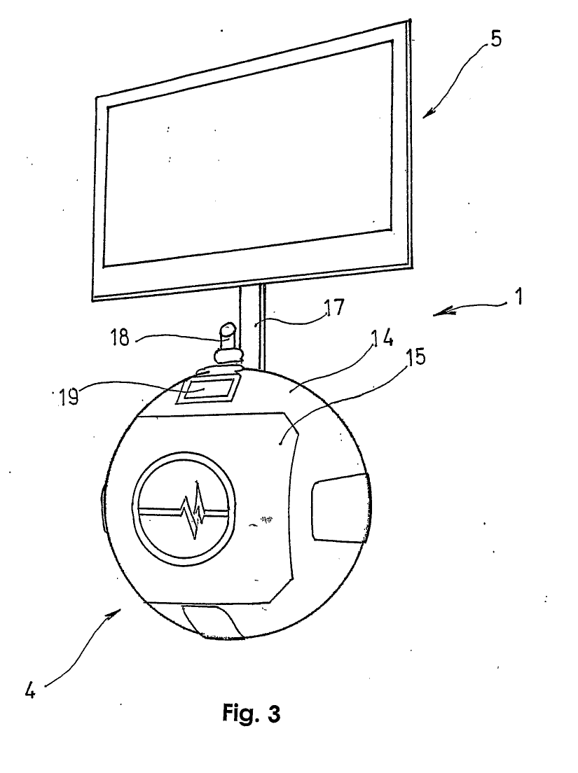

FIG. 3 is the perspective view of another possible construction of the installed unit;

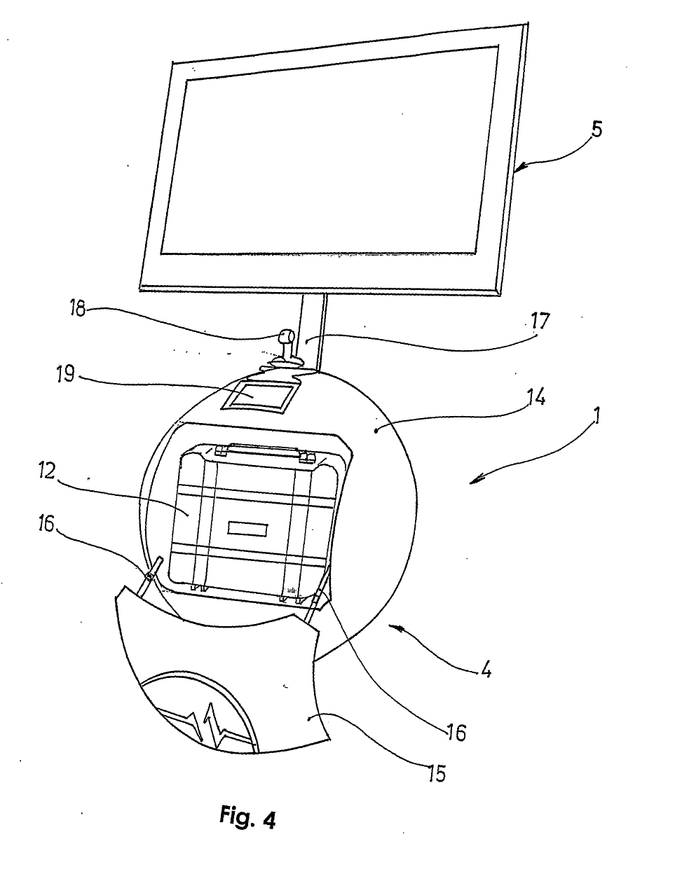

FIG. 4 shows the house of the block of the installed unit as in FIG. 3, with its door open;

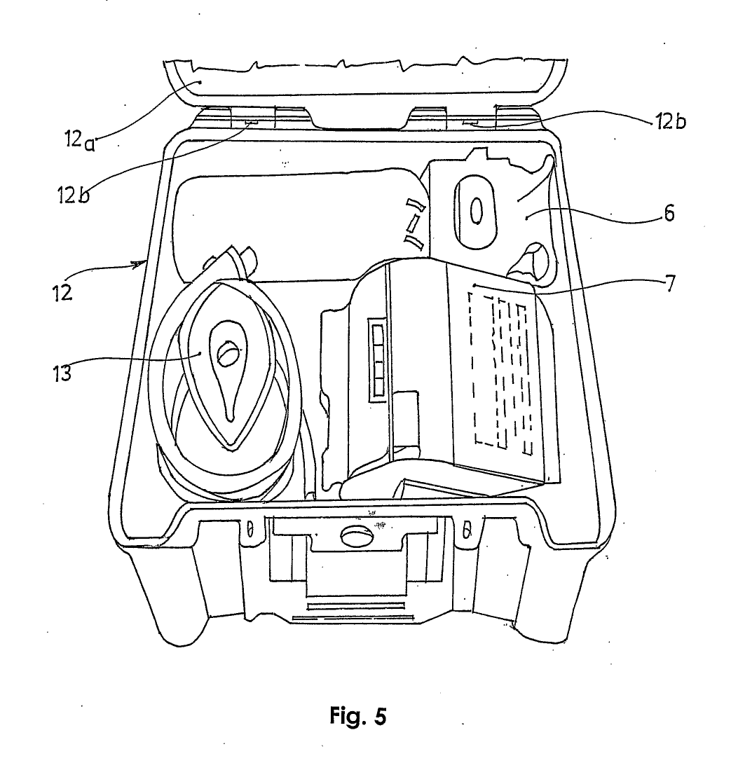

FIG. 5 is the perspective view of the inside of the bag situated in the house, on a larger scale, showing the first-aid tools inside it.

The installed units 1 are connected to the centre 2, which connection means that information is mediated practically in both directions. Furthermore the centre 2 is connected to a mobile rescue unit 3, in which case again connection means that information is mediated in both directions. It is obvious for specialists that in real life the number of the installed units 1, centres 2 and rescue units 3 can be significantly different from the number shown in the drawings. The number of the installed units is determined by the operator, and in a technical sense this number has no top limit, and it is also obvious that favourably there should be as many rescue units as possible included in the system according to the invention. It is also pointed out that on the one part the centre 2 can consist of units situated far away from each other, and an arrangement where the same rescue unit belongs to several independent centres is also within the scope of protection of the invention.

The installed units 1 may be permanently connected to the centre 2, or it is also possible to get connected to the centre by activation. In the case of permanent connection the centre may receive information continuously about the environment of the given installed unit, for example via still or motion pictures, or about the current status of the individual first-aid tools of the installed unit. The method of establishing connection by activation can be used, when maintaining connection continuously would occupy the resources of the centre unnecessarily, because there is no need for observing or monitoring the installed unit 1. Activation can take place for example as a result of the operation of any of the first-aid tools of the installed unit 1, by operating a switch constructed especially for this purpose, or for example in a given case by operating an emergency telephone set up in the installed unit 1.

If the centre 2 receives information from an installed unit 1, which information may require a doctor or a similar specialist, on the basis of a previously determined rule the centre 2 forwards this information to the appropriate place. Such a rule may be for example that the centre 2 must send the information to the ambulance station situated the closest to the given installed unit 1 or to the nearest rescue unit 3 (ambulance car). The nearest rescue unit 3 can be determined by comparing the actual positions of the operated installed unit 1 and the rescue units 3 forming a part of the system according to the invention, using the known state-of-the-art technology. For example at a given time the actual position of the rescue units 3 can be determined with the help of a state-of-the-art Global Positioning System (GPS). Such location data (e.g.: co-ordinates) is stored by the centre 2 on the basis of data sent by the individual rescue units 3 at regular intervals (e.g.: every minute). The centre 2 can obtain the location data of the operated installed unit 1 either from a previously stored database or from the data sent by the installed unit 1 during its operation. So by comparing the current location data of the installed unit 1 and the rescue units 3 the centre 2 determines the rescue unit 3 situated the locates to the installed unit 1 and sends information to it about that the installed unit 1 has been put into operation.

According to the example the centre 2 forwards the necessary information to the rescue unit 3. Such information may be for example the exact location of the given installed unit 1, which is an address, co-ordinates, and/or the input data of the route-planning program installed in the rescue unit 3. Furthermore, favourably the centre 2 forwards other information too about the given installed unit 1, for example still or motion pictures about the patient to be treated, or information about the patient's blood pressure, ECG, blood type, etc. As a result of this, the rescue unit 3 may obtain important information about the condition of the patient to be treated while it is on its way to the installed unit 1.

According to a favourable construction of the invention the rescue unit 3 can also forward information to the installed unit 1 via the centre 2. Such information may include for example instructions given to the persons on the site about what they need to do to stabilise the patient's condition. On the other hand the rescue unit 3 can provide directions in connection with the use of the first-aid tools of the installed unit 1, which is especially efficient, if the rescue unit 3 receives picture information from the site and so the staff of the rescue unit 3 can see the intervention in process and they can intervene verbally, if necessary.

FIG. 2 shows the schematic view of a possible construction of the installed unit 1. At the bottom of the figure the portable first aid device marked with reference number 4 suitable for the application of first aid and life saving contains first-aid tools and a few information mediating devices; the display unit, that is the information mediating device, formed by a flat screen in this case is marked with reference number 5. The installed unit 1 according to the example contains a breathing apparatus 6 and a defibrillator 7 as first-aid tools, and a screen 8, a camera 9, a telephone 10 and a GPS based position finder 11 as information mediating devices. The devices marked with reference numbers 6, 7, 8, 9, 10 and 11 together can be lifted out from the installed unit 1 in a bag 12 and inside this bag 12 they can all be taken directly to the patient in need of treatment. Between the portable first aid device 4 and the display unit 5 there is a data cable 13 to establishing operating connection. Practically the breathing apparatus 6 is an automatic breathing apparatus known in trade by the name “Oxylator FR300”. The defibrillator 7 can be of a construction or type from which information can be supplied directly about the condition of the person suffering an accident or taken by an indisposition, e.g.: ECG or blood pressure results, etc.

Practically it should be made possible to lift out the first-aid tools and information mediating devices in a bag 12, because generally the patient in need of treatment is not situated directly next to the installed unit 1 but further away, maybe hundreds of meters away. For this reason the installed unit 1 according to the example has a larger information mediating device, a display unit 5, which forms a fixed part of the installed unit 1, and also a smaller screen 8 forming a part of the portable bag 12.

The breathing apparatus 6 and defibrillator 7 according to the example are the most frequently needed first-aid—or life-saving—tools, but special knowledge is needed to operate them. For this reason, according to a favourable construction of the invention the bag 12 taken directly to the patient in need of treatment contains a GPS based position finder 11 to enable to arriving mobile rescue unit 3 to find the patient in need of treatment quickly (as it has been pointed out above, via the information mediating devices the installed unit 1 can also forward the exact location of the installed unit 1, or more precisely the exact location of the bag 12 of the installed unit 1, to the rescue unit 3 through the centre 2). Furthermore the bag 12 contains a screen 8, a camera 9 and a telephone 10 to ensure continuous picture- and sound-based contact with the rescue unit 3. Information between the bag 12 and the rescue unit 3 can be mediated through the centre 2, with the help of a state-of-the-art wired, wireless or combined wired and wireless data transfer system known in itself. The bag 12 according to the example also contains a breathing apparatus 6 and a defibrillator 7 used by the persons attending to the patient on the basis of the instructions received from the rescue unit 3, and such use can be monitored by the staff of the rescue unit 3 on the basis of the mediated picture and sound information. In a given case the operation of the breathing apparatus 6 and defibrillator 7 can also be displayed on the display unit 5.

Beside the rescue unit 3 the persons in the environment of the installed unit 1 can also receive information about what they should do from anyone else who is entitled to provide such information in the system, for example from the dispatcher of the centre 2. On the other hand the persons attending to the patient can also receive information automatically about what they should do. For example the installed unit 1 can be programmed in such a way that when a first-aid tool is removed from the installed unit 1, a series of figures or a film is shown on the screen 5 and/or 8 about how to operate the first-aid unit.

When using the breathing apparatus 6 and the defibrillator 7, they need to be operated in a harmonised way. For this reason, according to a favourable construction of the invention, the installed unit 1 monitors the operation parameters of the two first-aid devices in question and in a given case the patient's parameters too (e.g.: respiration, ECG, etc.), and on one of its information mediating devices, favourable on the flat screen 5 and/or on the screen 8 it displays instructions about which first-aid device needs to be used in the given moment and with what parameters.

The display unit 5 has two basic functions. On the one part, while attending to the patient the information relating to the treatment can be displayed on it in appropriate detail, as the screen 8 of the bag 12 is significantly smaller due to its portable nature, so the information displayed on it is less detailed. Therefore for example figures, series of figures, films showing the operation of the breathing apparatus 6 and the defibrillator 7 can be displayed on the display unit 5.

The display unit 5 has another important possible application too. When the installed unit is not used by anyone for applying first aid, advertisements, announcements or other information, in a given case public information, can be displayed on this screen. This possible application of the display unit 5 ensures the economical operation of the system according to the invention and makes this form of high-standard application of first-aid ensured by the invention easily accessible to anyone. Furthermore, when equipped with an appropriate data input device, e.g.: a keyboard, the display unit 5 can also be used as a public Internet access point.

It is obvious for specialists that the first-aid tools and information mediating devices placed in the installed units can be used in an extremely wide range of different combinations. Even installed units monitored by the same centre can be different from each other in respect of the various devices integrated in them.

The first-aid system according to the invention shown in FIGS. 1 and 2 is operated as described below:

during the periods between accidents/indispositions occurring in the environment of the installed unit 1—which periods cover most of the operation of the system, as accidents occur relatively rarely, and the application of first aid takes a relatively short time—, when the installed unit 1 is not used for applying first aid, advertisements, announcements, public announcements are mediated on its display unit, which is practically a flat plasma screen and, in a given case, on further screens or/and through loudspeakers belonging to the installed unit 1. As the installed unit 1 is situated on a busy public area—due to its intended use it is supposed to catch the attention of as many people as possible, e.g.: at railway stations, airports, busy public areas, etc.—, its location is absolutely favourable for the purpose of advertising and providing information, and so advertising can be ensured at optimal efficiency, and the display unit 5 can be expected to be used continuously by companies that intend to advertise themselves or their goods/services.

When an accident or a case of indisposition takes place in the environment of the installed unit 1, the person (or persons) entrusted to operate the installed unit 1 switches over the display unit 5 from advertising mode to first-aid mode, in other words suspends the display of advertisements and information and reports the accident immediately to the centre 2 and also to the ambulance service favourably at the same time. Then the person entrusted to operate the installed unit 1—an amateur life-saver, e.g.: a person working for an institute situated near the installed unit, a security guard, a traffic safety specialists, etc. or another suitable person trained to use the life-saving equipment, especially the breathing apparatus 6 and the defibrillator 7—applies first aid as needed in the given case on the basis of the film displayed on the display unit 5 showing the exact process of life-saving or the method of attending to any other problem, such as an injury, fracture, dislocation, etc., and on the basis of the instructions given by the doctor at the centre, if necessary, for example on the telephone, before the ambulance arrives. When the ambulance has taken away the patient, the operator mentioned above returns the tools used for applying first aid into the installed unit 1—obviously after replacing all used elements, e.g.: bandages, medicines with new ones; for example the battery of the defibrillator, the sucking discs and the oxygen tank must be replaced after each use—, locks the installed unit 1 and continues to display advertisements, announcements and information on the screen or display unit 5.

Another possible construction of the installed unit 1 is shown in detail in FIGS. 3-5. Here the portable first-aid device 4 as in FIG. 2 is placed in a spherical house 14, which is connected to the display unit 5 formed by a large flat plasma screen with the help of a stand 17, inside which there is a data cable 13 mentioned above, shown in FIG. 2. The house 14, which may stand on legs (not shown), has a door 15, which is in closed position in FIG. 3 and in open position in FIG. 4; according to this example opening and closing is performed by tilting the door 15 attached with connection elements 16, e.g.: hydraulic or pneumatic working cylinders, downwards or upwards. At the top a camera 18 is attached to the house 14 at the bottom of the stand 17, and above the door 15 or door opening there is a smaller display unit 19, which can practically be a touch-screen; the operating platform is situated here.

In FIG. 5 the bag 12 of the device 4 shown in FIG. 4 can be seen in open position, that is its top 12a has been turned upwards around the hinges 12b. Inside the bag 12 the defibrillator 7, the breathing apparatus 6—with the mask 13 belonging to it, used when applying first aid for placing it on the face of the person in need of treatment in a way known in itself—are shown on a larger scale.

The installed unit 1 shown in FIGS. 3-5 is designed to fill a more restricted function as compared to that of the one shown in FIGS. 1-2, as the bag 12 contains only a defibrillator 7 and a breathing apparatus 6, because it is basically designed for life-saving, namely for restoring the two basic life functions of an unconscious person—breathing and circulation—, that is for resuscitation. In accordance with this the installed unit 1 shown in FIGS. 3-5 can be established at a significantly lower cost than the one shown in FIGS. 1 and 2.

Obviously the house 14 itself is equipped with a camera 18—even when the bag 12 does not have one separately—, as it has extremely important functions: an immediate connection can be established between the doctor on duty and the patient situated near the installed unit 1; the doctor's image can appear on the screen, and audio-visual communication can be established immediately between the patient and the doctor; in a given case, with the help of the camera the doctor can follow the whole process of resuscitation, act as an assistant, give instructions, etc. Apart from the above the camera also has important additional functions, such as public are monitoring; human contact number monitoring; disaster prevention, etc.

Typically the installed unit 1 as in FIGS. 3-5 is set up and operated on areas, where there is a large number of people permanently or frequently, for example at railway stations, shopping centres, busy city centres, sports facilities, etc. An operating staff is allocated to each installed unit 1, the members of the staff are trained to use the life-saving devices, but they are not necessarily health specialists, they can be for example security guards, information desk assistants, members of the local staff, etc. Those who complete the necessary training course may be given an electronic card with an identifier on it, and with the use of this card the door 15 of the house 14 (FIGS. 3 and 4) can be opened. In the case of emergency—after the person having the card has opened the door 15 and taken out the bag 12—at least three telephone calls need to be initiated, namely
the ambulance service is called,
the centre of the system is called,
the system maintenance service is called, because after each use the battery of the defibrillators, the sucking discs and the oxygen tank need to be replaced.

It is pointed out here that the same person may also be responsible for providing advertisements and information on the basis of the instructions received from the centre.

Obviously when the door 15 is opened, the displaying of advertisements and information is suspended; in this case on the display unit 5, practically a plasma screen, LCD screen or touch-screen, a film can be seen showing the exact process of life saving to provide help for the amateur life saver.

The basic advantage of the invention is that it is able to provide life-saving interventions of maximum efficiency and other first-aid application tasks in the form of a public service for many people in such a way that the financial conditions needed for establishing and operating the system, or at least a part of them, are realised due to the system itself, by displaying advertisements, announcements, informations etc.

Obviously the invention is not restricted to the possible solutions described above, but it can be realised in several different ways within the scope of protection defined in the claims.

1: First aid application system, which has at least one installed unit (1) containing first-aid tools and a device (devices) for forwarding information, which installed unit (1) is in communicating connection with a centre (2) receiving and forwarding information concerning first aid application, characterised by that
the information mediating device(s) of the installed unit (1) is (are) constructed to be suitable for displaying advertisements, announcements or other, for example public information other than first aid information, or/and they also contain a tool for this purpose,
the installed unit (1) has at least a defibrillator (7) and a breathing apparatus (6) as first-aid tools.

2: System as in claim 1, characterised by that the centre (2) is in communicating connection with several installed units (1).

3: System as in claim 1, characterised by that as an information mediating device—favourably equipped with loudspeakers—it has a display unit (5; 8), practically a screen, favourably a flat screen, especially favourably a plasma screen or LCD screen or a touch-screen; and/or a camera (9), favourably a digital camera, especially favourably a CCD camera, a surveillance camera, and/or a wired or wireless telephone (10); and/or a microphone, especially a head microphone or outdoor monitoring microphone; and/or a loudspeaker; and/or a position finder (11), especially a position finder based on a Global Positioning System (GPS).

4: System as in claim 1, characterised by that as first-aid tools the installed unit (1) contains bandages and/or a disinfectant; and/or EKG recorder; and/or blood pressure monitor; and/or a blood typing kit; and/or medicines, especially analgesics; and/or tools for cutting, especially scissors or/and a scalpel.

5: System as in claim 1, characterised by that its centre (2) is constructed to be suitable for receiving, forwarding, processing and—in a given case—storing information.

6: System as in claim 1, characterised by that the installed unit (1) has an information mediating device (devices) suitable for forwarding information automatically.

7: System as in claim 1, characterised by that the information mediating device(s) of the installed unit (1) is (are) constructed to be suitable for providing information regarding the use of the first-aid tools.

8: System as in claim 1, characterised by that the installed unit (1) contains a device (devices) suitable for mediating information, advertisements, emergency warnings and other announcements—practically from a centre—locally, within a given action radius in the environment of the installed unit, to mobile telephones, PDAs and similar devices, for example via SMS, MMS, etc.

9: System as in claim 1, characterised by that the installed unit (1) contains a device providing WiFi access in its environment, within a given action radius.

10: Procedure for the operation of the system defined in claim 1, characterised by that
in the case of an accident or/and indisposition information is forwarded from the installed unit (1) to the centre (2) about the event;
on the basis of the information received from the centre (2) the person(s) suffering an accident or taken by an indisposition is (are) attended to—applied first aid—on the site, if necessary;
during the period between the application of first aid and the next similar application advertisements or other information is provided continuously or with interruptions using the installed unit.

11: Procedure as in claim 10, characterised by that advertisements or other information is mediated using the same information forwarding device, especially flat screen, favourably plasma screen, LCD screen or touch-screen as used for displaying information concerning the application of first aid.

12: Procedure as in claim 10, characterised by that in the case of life-saving interventions defibrillation and artificial respiration is applied at the same time.

13: Procedure as in claim 10, characterised by that information is forwarded from the site of the accident/indisposition to the centre about the site of the accident and the patient's condition, and, in a given case, about the patient's age, sex, one or more identifiers; and still or motion pictures are mediated about the patient or/and about a part—e.g.: an injured part—of the patient's body; and, if necessary, information is provided about the patient's blood type, blood pressure and ECG recording.

14: Procedure as in claim 10, characterised by that information is forwarded from the centre or through the centre to the site of the accident especially about stabilising the patient's condition and the method of using the available first-aid tools, mainly the defibrillator and the breathing apparatus—especially “Oxylator FR300” type, favourably automatic breathing apparatus—together or simultaneously.

15: Procedure as in claim 10, characterised by that a rescue unit is sent from the centre or through the centre to the site of the accident/indisposition, which rescue unit is provided with the information received from the site of the accident needed for the treatment of the patient.

16: Portable first-aid device for use especially in the first-aid system as in claim 1, which has devices for the application of first aid situated in the storing-carrying unit, characterised by that it contains at least a breathing apparatus (6) and a defibrillator (7).

17: Device as in claim 16, characterised by that it has automatic breathing apparatus (6).

18: Device as in claim 16, characterised by that the defibrillator (7) is constructed to be suitable for forwarding information concerning the patient's condition especially to a place where ECG tests are performed and the test results are evaluated.
